(19) United States
(12) Patent Application Publication
(10) Pub. No.: US 2019/0000421 A1
(43) Pub. Date: Jan. 3, 2019

(54) THREE-DIMENSIONAL IMAGING ULTRASONIC SCANNING METHOD

(71) Applicant: Telefield Medical Imaging Limited, Hong Knog (HK)

(72) Inventor: Yongping Zheng, Hong Kong (HK)

(73) Assignees: Telefield Medical Imaging Limited, Hong Kong (HK); Telefield Medical Imaging Limited, Hong Kong (HK)

(21) Appl. No.: 15/748,633

(22) PCT Filed: Apr. 8, 2016
(86) PCT No.: PCT/CN2016/078835

§ 371 Date: Jan. 29, 2018

(30) Foreign Application Priority Data
Jul. 28, 2015 (CN) 201510452165.7

(51) Int. Cl. A61B 8/00; A61B 8/08

(52) U.S. Cl. CPC A61B 8/466; A61B 8/4461; A61B 8/5207; A61B 8/4477; A61B 8/4218

(57) ABSTRACT

A three-dimensional imaging ultrasonic scanning method applicable to ultrasonic diagnostic instruments is disclosed. The three-dimensional imaging ultrasonic scanning method can simultaneously satisfy different requirements for images during three-dimensional ultrasonic scanning, so that with just one time of scanning, a series of B-mode ultrasound images corresponding to each of the ultrasonic arrays can be obtained, and further three-dimensional images for the tested object can be constructed by a main body portion of ultrasonic diagnosis, laying a better foundation for ultrasonic diagnosis.

### TECHNICAL FIELD

The present application relates to the technical field of the ultrasonic scanning used in ultrasonic diagnostic instruments, and more particularly relates to a three-dimensional imaging ultrasonic scanning method.

### BACKGROUND

At present, in the ultrasonic diagnostic instruments, the three-dimensional ultrasound imaging can be accomplished by moving the B-ultrasound probe to a series of spatial positions for recording the B-ultrasound images (2D) at these spatial positions and reconstructing the three-dimensional ultrasound images based on the simultaneously recorded B-ultrasound images (2D). However, in the existing technology just a single one B-ultrasound probe is moved to do this, that is, the resulting ultrasound image is completely determined by the characteristics of the single one B-ultrasound probe, among them, which characteristics include ultrasound frequency, resolution, penetration depth, image width, image shape, focus mode, and image direction. However, in the three-dimensional ultrasound scanning, different organizations, different parts, different patients, different requirements, often have different requirements for these parameters, and meanwhile different requirements may need to be satisfied. For example, in the three-dimensional ultrasound imaging of the spine, an ultrasound probe with a higher frequency should be employed for the muscle tissue which may need high resolution, while an ultrasound probe with a lower frequency should also be employed for the deep tissue imaging. In this case, using a single one ultrasonic probe for the three-dimensional ultrasound imaging cannot meet the actual requirements.

### SUMMARY

The object of the present application is to provide a three-dimensional imaging ultrasonic scanning method, aiming at the above defects of the prior art that a single one ultrasonic probe cannot satisfy the different requirements of the three-dimensional imaging in the three-dimensional ultrasonic scanning at the same time.

In one aspect, a three-dimensional imaging ultrasonic scanning method is provided for solving above technical problem, which including following steps:

S0, generating a high-frequency voltage pulse for driving a plurality of ultrasonic arrays and powering a spatial locator to operate;

S1, acquiring different ultrasonic image information of a tested object by the plurality of ultrasonic arrays;

S2, acquiring positional information of the plurality of ultrasonic arrays by the spatial locator.

Advantageously, in the step S1, the plurality of ultrasonic arrays implement a scanning at the same time, or at different times or at fixed relative positions.

Advantageously, in the step S2, the spatial locator is a positioner based on electromagnetic field measurements.

Advantageously, in the step S2, the spatial locator is a motor driving device with a positioning function, wherein the plurality of ultrasonic arrays are mounted on the motor driving device.

Advantageously, the motor driving device with the positioning function is a linear scanning device, wherein the plurality of ultrasonic arrays are mounted on the linear scanning device.

Advantageously, the motor driving device with the positioning function is a circular scanning device, wherein the plurality of ultrasonic arrays are mounted on the circular scanning device.

Advantageously, the plurality of ultrasonic arrays have different frequencies/sizes/focus modes/shapes/mounting orientations.

Advantageously, the plurality of ultrasonic arrays include at least a first ultrasound array and a second ultrasound array, wherein, the first ultrasonic array and the second ultrasonic array have the same mounting orientation but different frequencies for acquiring different image information of a same part of the tested object.

Advantageously, the first ultrasonic array is arranged as a linear array while the second ultrasonic array is arranged as an arc-shaped array for acquiring different scanning ranges of the tested object.

Advantageously, the three-dimensional imaging ultrasonic scanning method of the present application further includes a following step:

S3, reconstructing a three-dimensional image of the tested object based on the different ultrasonic image information of the tested object and the positional information of the plurality of ultrasonic arrays.

The three-dimensional imaging ultrasound scanning method of the present invention employs at least two ultrasound B-ultrasound arrays having different parameters so that with just one time of scanning, a series of B-mode ultrasound images corresponding to each of the ultrasonic arrays can be obtained. According to the actual needs, the series of images meeting the requirements can be selected for the image three-dimensional ultrasound imaging. For example, two ultrasonic arrays with different frequencies are mounted in parallel, such that images obtained from the ultrasonic arrays with a higher frequency may be used for the superficial tissues to obtain a higher image resolution, while images obtained from the ultrasonic arrays with a lower frequency may be used for the deeper tissues to ensure a sufficient penetration depth. Two ultrasound probes with different shapes may be used, for example one of which can be a linear array and the other one can be an arc-shaped array. A higher resolution may be obtained by the linear array, while a larger scanning range may be obtained by the arc-shaped array. Different ultrasonic arrays can also be installed in different directions so that three-dimensional images of multiple interested regions or three-dimensional images of the same interested region in different directions, such as the images of the spinous processes in different directions, can be obtained by one time of three-dimensional scanning In addition to satisfying different requirements by selecting images from different series of ultrasound images obtained by the different ultrasonic arrays, the corresponding images in different series can also be used for image fusion processing to achieve higher image quality, for example, the high signal to noise ratio of the image, thus providing a good foundation for the ultrasound diagnosis.

### BRIEF DESCRIPTION OF THE DRAWINGS

FIG. 1 is a flowchart of the three-dimensional imaging ultrasonic scanning method according to a first embodiment of the present application.

FIG. 2 is a schematic diagram of the structure of the motor driving device with a positioning function in FIG. 1, wherein the motor driving device is a circular scanning device.

FIG. 3 is a flowchart of the three-dimensional imaging ultrasonic scanning method according to a second embodiment of the present application.

FIG. 4 is an external view of a preferred embodiment of the first ultrasonic array and the second ultrasonic array in FIG. 3.

FIG. 5 is an external view of another preferred embodiment of the first ultrasonic array and the second ultrasonic array in FIG. 3.

### DETAILED DESCRIPTION OF THE PREFERRED EMBODIMENT

The present application provides a three-dimensional imaging ultrasonic scanning method applicable to the ultrasonic diagnostic instruments. The three-dimensional imaging ultrasonic scanning method can simultaneously satisfy different requirements for images during three-dimensional ultrasonic scanning A specific solution is to simultaneously move at least two ultrasound B-ultrasound arrays with different parameters in a three-dimensional imaging scanning, so that a series of B-ultrasound images corresponding to each ultrasound array can be obtained in a single one time of scanning At the same time, combining with the spatial locator, the series of images meeting the requirements can be selected for the image three-dimensional ultrasound imaging. In addition to satisfying different requirements by selecting images from different series of ultrasound images obtained by the different ultrasonic arrays, the corresponding images in different series can also be used for image fusion processing to achieve higher image quality, for example, the high signal to noise ratio of the image, thus providing a good foundation for ultrasonic diagnosis.

To make the object, the technical solution, and the advantage of the present application more clearly, the present application is further described in detail below with reference to the accompanying drawings and embodiments. It should be understood that the specific embodiments described herein are merely used to explain the present invention and are not intended to the present application.

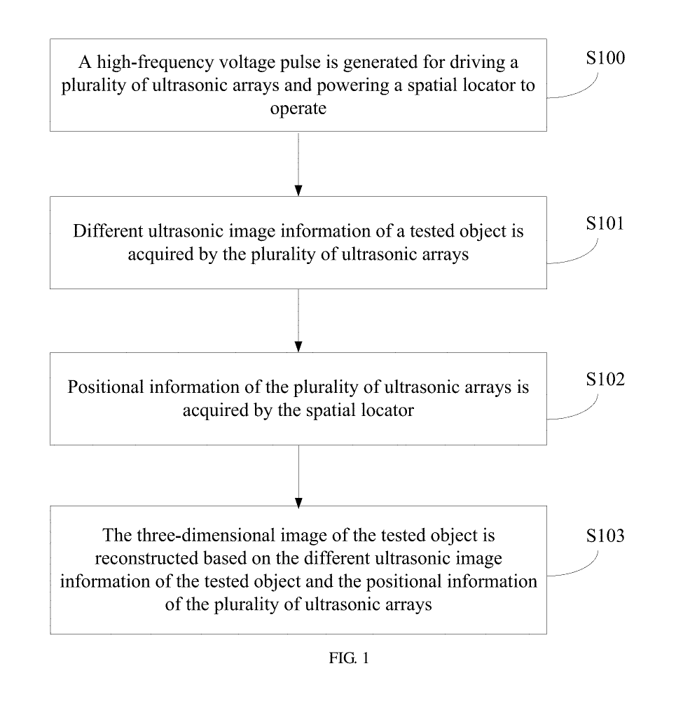

As shown in FIG. 1, a flowchart of the three-dimensional imaging ultrasonic scanning method according to a first embodiment of the present application is disclosed. The three-dimensional imaging ultrasonic scanning method comprises the following steps.

In step S100, a high-frequency voltage pulse is generated for driving a plurality of ultrasonic arrays and powering a spatial locator to operate.

In this step, the high-frequency voltage pulse is generated by a transmission circuit which is positioned in the ultrasonic diagnostic instrument.

In this embodiment, the transmission circuit can be composed of a clock generator, a frequency divider, a transmission delay circuit, and a pulse generator. The clock pulse generated by the clock generator is passed through the frequency divider to be lowered to a rate pulse with a certain frequency which is then passed through the transmission delay circuit to the pulse generator for generating a high frequency voltage pulse to drive the plurality of ultrasound arrays. That is, the transmission circuit transmits the electric signals to the plurality of ultrasonic arrays and drives the plurality of ultrasonic arrays, so that the plurality of ultrasonic arrays transmit the ultrasonic beams to the tested object, which belongs to the prior art and is not described herein again.

In step S101, different ultrasonic image information of a tested object is acquired by the plurality of ultrasonic arrays.

In this step, the plurality of ultrasonic arrays implement a scanning at the same time, or at different times or at fixed relative positions. During the scanning, the plurality of ultrasonic arrays respectively send ultrasonic waves to the tested object, receive the ultrasonic echo, and output corresponding electric signals according to the ultrasonic echo. Among them, the plurality of ultrasonic arrays have different frequencies, sizes, focus modes, shapes, mounting orientations, and the combinations thereof.

The above electrical signal is also needed to go through the amplifying circuit, the delay circuit and the addition circuit for further processing, so that the main part of the ultrasonic diagnostic instrument can better receive the electrical signal that represents the different information of the tested object. Wherein, the amplifying circuit is configured to perform low-noise amplification or buffering operation on the received or transmitted ultrasonic signals to better transmit the ultrasonic signals. The delay circuit and the addition circuit are respectively used to delay and add the electric signal of the ultrasonic wave.

In this embodiment, the ultrasonic arrays are arranged in different shapes to acquire images of different scanning ranges. The shapes of the ultrasonic arrays include a line array, an arc-shaped array, and a two-dimensional array. Wherein, an image of higher resolution is obtained when using the linear array for scanning, while an image of larger scanning range is obtained when using the arc-shaped array for scanning

In this embodiment, when the mounting orientations of the plurality of ultrasonic arrays are the same, different frequencies can be set, so that images of the same tested object at different depths can be obtained according to the different frequencies. When the mounting orientations of the plurality of ultrasonic arrays are different, images of multiple desired scanning areas or images of the same scanning area in different directions can be obtained by one time of scanning

In step S102, positional information of the plurality of ultrasonic arrays is acquired by the spatial locator.

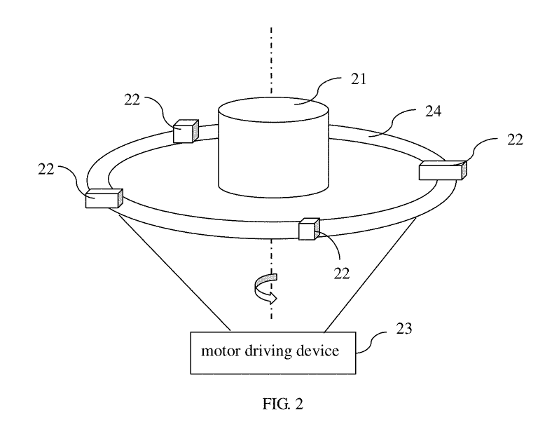

In this step, the positional information of the plurality of ultrasonic arrays during the scanning is acquired by the spatial locator. During the scanning, the electric signals outputted by the ultrasonic arrays and the corresponding positional information are outputted to the main part of the ultrasonic diagnostic instrument. According to the scanning process, the spatial locator locates the positional information of the plurality of ultrasonic arrays to transmit the positional information to the main part of the ultrasonic diagnostic instrument for image-related processing.

Wherein, the spatial locator may be a positioner based on electromagnetic field measurement or a motor driving device with a positioning function. When the spatial locator is the motor driving device with a positioning function, the plurality of ultrasonic arrays are mounted at corresponding positions according to different forms of the motor driving device with a positioning function. When the motor driving device with a positioning function is a linear scanning device, the plurality of ultrasonic arrays are mounted on the linear scanning device. When the motor driving device with a positioning function is a circular scanning device, the plurality of ultrasonic arrays are mounted on the circular scanning device. As shown in FIG. 2, the circular scanning device includes a motor driver 23 and a supporting body 24 driven by the motor driver 23 to rotate. The supporting body 24 has a circular shape. The tested object 21 is placed at the center of the supporting body 24. The plurality of ultrasonic arrays 22 are mounted on the supporting body 24 and equally spaced along the circumference of the supporting body 24.

In step S103, the three-dimensional image of the tested object is reconstructed based on the different ultrasonic image information of the tested object and the positional information of the plurality of ultrasonic arrays.

In this step, the main part of the ultrasonic vibration apparatus can acquire a scanned three-dimensional image based on the positional information acquired by the spatial locator and the electric signal of the different ultrasonic image information outputted by the ultrasonic arrays after image processing.

Specifically, a plurality of three-dimensional images of the tested object are reconstructed by performing image processing on the different ultrasonic image information of the tested object and the positional information of the plurality of ultrasonic arrays, wherein each three-dimensional image is reconstructed from the ultrasound image information obtained from each ultrasound array.

Alternatively, the three-dimensional image of the tested object is reconstructed by performing comprehensive image processing on the different ultrasonic image information of the tested object and the positional information of the plurality of ultrasonic arrays. Wherein, the three-dimensional image is obtained by reconstructing and fusing the different ultrasonic image information obtained by the plurality of ultrasonic arrays.

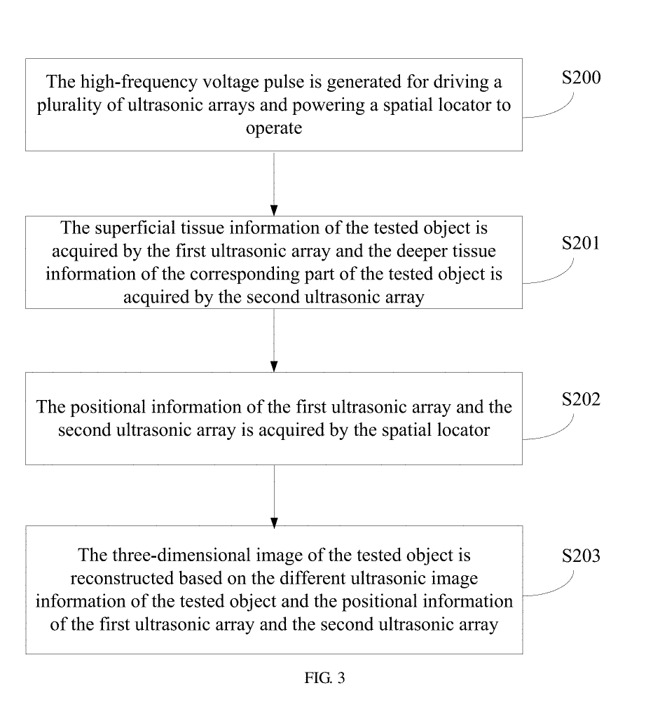

As shown in FIG. 3, a flowchart of the three-dimensional imaging ultrasonic scanning method according to a second embodiment of the present application is disclosed, in which the plurality of ultrasonic arrays include a first ultrasonic array and a second ultrasonic array.

In this embodiment, the method comprises the following steps. In step S200, the high-frequency voltage pulse is generated for driving a plurality of ultrasonic arrays and powering a spatial locator to operate.

In this step, the high-frequency voltage pulse is generated by a transmission circuit which is positioned in the ultrasonic diagnostic instrument.

In this embodiment, the transmission circuit can be composed of a clock generator, a frequency divider, a transmission delay circuit, and a pulse generator. The clock pulse generated by the clock generator is passed through the frequency divider to be lowered to a rate pulse with a certain frequency which is then passed through the transmission delay circuit to the pulse generator for generating a high frequency voltage pulse to drive the plurality of ultrasound arrays. That is, the transmission circuit transmits the electric signals to the plurality of ultrasonic arrays and drives the plurality of ultrasonic arrays, so that the plurality of ultrasonic arrays transmit the ultrasonic beams to the tested object, which belongs to the prior art and is not described herein again.

In step S201, the superficial tissue information of the tested object is acquired by the first ultrasonic array and the deeper tissue information of the corresponding part of the tested object is acquired by the second ultrasonic array.

In this step, the first ultrasonic array and the second ultrasonic array may implement a scanning at the same time, or at different times or at fixed relative positions. During the scanning, the first ultrasonic array and the second ultrasonic array respectively send ultrasonic waves to the tested object, receive the ultrasonic echo, and output corresponding electric signals according to the ultrasonic echo. Wherein, both the first ultrasonic array and the second ultrasonic array are linear arrays having the same mounting orientation but different frequencies.

In step S202, the positional information of the first ultrasonic array and the second ultrasonic array is acquired by the spatial locator.

In this step, the positional information of the first ultrasonic array and the second ultrasonic array during the scanning is acquired by the spatial locator. During the scanning, the electric signals outputted by the ultrasonic arrays and the corresponding positional information are outputted to the main part of the ultrasonic diagnostic instrument. According to the scanning process, the spatial locator locates the positional information of the plurality of ultrasonic arrays to transmit the positional information to the main part of the ultrasonic diagnostic instrument for image-related processing.

Wherein, the spatial locator may be a positioner based on electromagnetic field measurement or a motor driving device with a positioning function. When the spatial locator is the motor driving device with a positioning function, the first ultrasonic array and the second ultrasonic array are mounted at corresponding positions according to different forms of the motor driving device with a positioning function. When the motor driving device with a positioning function is a linear scanning device, the first ultrasonic array and the second ultrasonic arrays are mounted on the linear scanning device. When the motor driving device with a positioning function is a circular scanning device, the first ultrasonic array and the second ultrasonic array are mounted on the circular scanning device.

In step S203, the three-dimensional image of the tested object is reconstructed based on the different ultrasonic image information of the tested object and the positional information of the first ultrasonic array and the second ultrasonic array.

In this step, the main part of the ultrasonic vibration apparatus can acquire a scanned three-dimensional image based on the positional information acquired by the spatial locator and the electric signals for the same part of the tested object at the different depths outputted by the first ultrasonic array and the second ultrasonic array after image processing.

Specifically, two three-dimensional images of the tested object are reconstructed by performing image processing on the different ultrasonic image information of the tested object and the positional information of the first ultrasonic array and the second ultrasonic array, wherein one three-dimensional image is reconstructed from the ultrasound image information obtained from the first ultrasound array, and the other three-dimensional image is reconstructed from the ultrasound image information obtained from the second ultrasound array.

Alternatively, the three-dimensional image of the tested object is reconstructed by performing comprehensive image processing on the different ultrasonic image information of the tested object and the positional information of the first ultrasonic array and the second ultrasonic array. Wherein, the three-dimensional image is obtained by reconstructing and fusing different ultrasonic image information obtained by the first ultrasonic array and the second ultrasonic array.

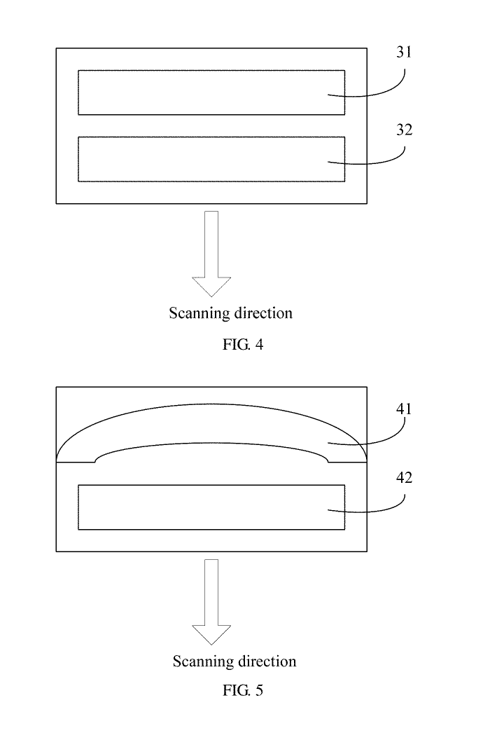

As shown in FIG. 4, an external view of a preferred embodiment of the first ultrasonic array and the second ultrasonic array in FIG. 3 is disclosed. In this embodiment, both the first ultrasonic array 31 and the second ultrasonic array 32 are linear arrays arranged in parallel, that is, the mounting orientations of the first ultrasonic array 31 and the second ultrasonic array 32 are the same. During the scanning, the areas scanned by the first ultrasonic array 31 and the second ultrasonic array 32 are the same. The frequency of the first ultrasonic array 31 is f₀, and the frequency of the second ultrasonic array 32 is f₁. When f₀ and f₁ are not equal, the images of the same tested object at the different depths can be obtained according to the different frequencies. In the present application, the mounting orientations of the first ultrasonic array 31 and the second ultrasonic array 32 are not limited to this. In actual use, the ultrasonic arrays may adopt other mounting orientations. Meanwhile, other parameters of the ultrasonic arrays, such as the shape and the size, may also be different according to practical needs.

As shown in FIG. 5, an external view of another preferred embodiment of the first ultrasonic array and the second ultrasonic array in FIG. 3 is disclosed. In this embodiment, the first ultrasonic array 41 is arranged as a linear array, while the second ultrasonic array 42 is arranged as an arc-shaped array. The mounting orientations of the first ultrasonic array 41 and the second ultrasonic array 42 are the same. During scanning, a higher resolution is obtained by the scanning of the first ultrasonic array 41, and a larger scanning area is obtained by the scanning of the second ultrasonic array 42. In the present application, the mounting orientations of the first ultrasonic array 41 and the second ultrasonic array 42 are not limited to this. In actual use, the ultrasonic arrays may adopt other mounting orientations. Meanwhile, other parameters of the ultrasonic arrays, such as the shape and the size, may also be different according to practical needs.

In summary, the present application provides a three-dimensional imaging ultrasonic scanning method applicable to ultrasonic diagnostic instruments. The three-dimensional imaging ultrasonic scanning method can simultaneously satisfy different requirements for images during three-dimensional ultrasonic scanning A specific solution is to simultaneously move at least two ultrasound B-ultrasound arrays with different parameters in a three-dimensional imaging scanning Combining with the spatial locator, a series of B-ultrasound images corresponding to each ultrasound array can be obtained in a single one time of scanning, so that the main part of the ultrasound diagnosis can construct a three-dimensional image of the tested object, thus providing a good foundation for the ultrasound diagnosis.

As described above, it is only a better specific implementation method of the application, but the scope of protection of the application is not limited to this. Any variation or replacement that can be easily thought of by persons skilled in the art within the technical scope disclosed by the present application shall fall within the protection scope of the present application. Therefore, the protection scope of the present application should be subject to the protection scope of the claims.

1-10. (canceled)

11. A three-dimensional imaging ultrasonic scanning method applicable to ultrasonic diagnostic instruments, including following steps:
S0, generating a high-frequency voltage pulse for driving a plurality of ultrasonic arrays and powering a spatial locator to operate;
S1, acquiring different ultrasonic image information of a tested object by the plurality of ultrasonic arrays;
S2, acquiring positional information of the plurality of ultrasonic arrays by the spatial locator.

12. The three-dimensional imaging ultrasonic scanning method according to claim 11, wherein in the step S1, the plurality of ultrasonic arrays implement a scanning at the same time, or at different times or at fixed relative positions.

13. The three-dimensional imaging ultrasonic scanning method according to claim 11, wherein, in the step S2, the spatial locator is a positioner based on electromagnetic field measurements.

14. The three-dimensional imaging ultrasonic scanning method according to claim 11, wherein in the step S2, the spatial locator is a motor driving device with a positioning function, wherein the plurality of ultrasonic arrays are mounted on the motor driving device.

15. The three-dimensional imaging ultrasonic scanning method according to claim 14, wherein the motor driving device with the positioning function is a linear scanning device, wherein the plurality of ultrasonic arrays are mounted on the linear scanning device.

16. The three-dimensional imaging ultrasonic scanning method according to claim 14, wherein the motor driving device with the positioning function is a circular scanning device, wherein the plurality of ultrasonic arrays are mounted on the circular scanning device.

17. The three-dimensional imaging ultrasonic scanning method according to claim 11, wherein the plurality of ultrasonic arrays have different frequencies/sizes/focus modes/shapes/mounting orientations.

18. The three-dimensional imaging ultrasonic scanning method according to claim 12, wherein the plurality of ultrasonic arrays have different frequencies/sizes/focus modes/shapes/mounting orientations.

19. The three-dimensional imaging ultrasonic scanning method according to claim 13, wherein the plurality of ultrasonic arrays have different frequencies/sizes/focus modes/shapes/mounting orientations.

20. The three-dimensional imaging ultrasonic scanning method according to claim 14, wherein the plurality of ultrasonic arrays have different frequencies/sizes/focus modes/shapes/mounting orientations.

21. The three-dimensional imaging ultrasonic scanning method according to claim 15, wherein the plurality of ultrasonic arrays have different frequencies/sizes/focus modes/shapes/mounting orientations.

22. The three-dimensional imaging ultrasonic scanning method according to claim 16, wherein the plurality of ultrasonic arrays have different frequencies/sizes/focus modes/shapes/mounting orientations.

23. The three-dimensional imaging ultrasonic scanning method according to claim 17, wherein the plurality of ultrasonic arrays have different frequencies/sizes/focus modes/shapes/mounting orientations.

24. The three-dimensional imaging ultrasonic scanning method according to claim 17, wherein the plurality of ultrasonic arrays include at least a first ultrasound array and a second ultrasound array, wherein, the first ultrasonic array and the second ultrasonic array have the same mounting orientation but different frequencies for acquiring different image information of a same part of the tested object.

25. The three-dimensional imaging ultrasonic scanning method according to claim 18, wherein the plurality of ultrasonic arrays include at least a first ultrasound array and a second ultrasound array, wherein, the first ultrasonic array and the second ultrasonic array have the same mounting orientation but different frequencies for acquiring different image information of a same part of the tested object.

26. The three-dimensional imaging ultrasonic scanning method according to claim 19, wherein the plurality of ultrasonic arrays include at least a first ultrasound array and a second ultrasound array, wherein, the first ultrasonic array and the second ultrasonic array have the same mounting orientation but different frequencies for acquiring different image information of a same part of the tested object.

27. The three-dimensional imaging ultrasonic scanning method according to claim 20, wherein the plurality of ultrasonic arrays include at least a first ultrasound array and a second ultrasound array, wherein, the first ultrasonic array and the second ultrasonic array have the same mounting orientation but different frequencies for acquiring different image information of a same part of the tested object.

28. The three-dimensional imaging ultrasonic scanning method according to claim 21, wherein the plurality of ultrasonic arrays include at least a first ultrasound array and a second ultrasound array, wherein, the first ultrasonic array and the second ultrasonic array have the same mounting orientation but different frequencies for acquiring different image information of a same part of the tested object.

29. The three-dimensional imaging ultrasonic scanning method according to claim 24, wherein the first ultrasonic array is arranged as a linear array while the second ultrasonic array is arranged as an arc-shaped array for acquiring different scanning ranges of the tested object.

30. The three-dimensional imaging ultrasonic scanning method according to claim 11, wherein further including a following step:
S3, reconstructing a three-dimensional image of the tested object based on the different ultrasonic image information of the tested object and the positional information of the plurality of ultrasonic arrays.
